(19) United States
(12) Patent Application Publication
(10) Pub. No.: US 2011/0287958 A1
(43) Pub. Date: Nov. 24, 2011

(54) Method for Using Gene Expression to Determine Colorectal Tumor Stage

(76) Inventors: Steven Shak, Hillsborough, CA (US); Margarita Lopatin, Redwood City, CA (US); Mark Lee, Los Altos Hills, CA (US)

(21) Appl. No.: 13/111,814

(22) Filed: May 19, 2011

Related U.S. Application Data
(60) Provisional application No. 61/346,687, filed on May 20, 2010.

(51) Int. Cl. C40B 30/04; G01N 33/574; C12Q 1/02; C12Q 1/68; C12Q 1/37

(52) U.S. Cl. 506/9; 435/6.12; 435/23; 435/29; 435/7.1

(57) ABSTRACT

The invention relates to methods of determining a colorectal tumor stage in a patient by gene expression analysis. The method comprises assaying the level of at least one RNA transcript, or its expression product, which is correlated to colorectal tumor stage. The invention may be useful for determining whether a patient has stage II or stage III colorectal cancer.

This application claims priority to U.S. Provisional Application No. 61/346,687, filed May 20, 2010, which is incorporated by reference in its entirety.

### TECHNICAL FIELD

The present disclosure relates to molecular diagnostic assays that provide information concerning tumor stage in colorectal cancer patients. Specifically, the present disclosure provides certain genes, the expression levels of which may be used to determine tumor stage.

### INTRODUCTION

Colorectal cancer is the third most common malignant neoplasm worldwide, and the second leading cause of cancer-related mortality in the United States and the European Union. It is estimated that there will be approximately 150,000 new cases diagnosed each year in the United States, with about 65% of these being diagnosed as stage II/III colorectal cancer.

Clinical diagnosis of colorectal cancer generally involves evaluating the progression status of the cancer using standard classification criteria. Two classification systems have been widely used in colorectal cancer, the modified Duke's (or Astler-Coller) staging systems and more recently TNM staging as developed by the American Joint Committee on Cancer. Staging is the process of determining how far a cancer has spread based on clinical observation and pathologic examination of how far the primary tumor (T) has extended into the wall of the intestine and the extent of spread into regional lymph nodes (N) or distant metastasis (M).

Estimates of recurrence risk and treatment decisions in colorectal cancer are currently based primarily on tumor stage. Although tumor stage has been demonstrated to have significant association with outcome sufficient to be included in pathology reports, the College of American Pathologists Consensus Statement noted that variations in approach to the acquisition, interpretation, reporting, and analysis of this information exist. C. Compton, et al., Arch Pathol Lab Med 124:979-992 (2000). As a consequence, existing pathologic staging methods have been criticized as lacking reproducibility.

### SUMMARY

Molecular assays that involve measurement of expression levels of staging genes, and gene subsets, from a biological sample obtained from a cancer patient, and analysis of the measured expression levels to provide information concerning the stage of the tumor for the patient are provided herein. Methods of analysis of gene expression values of staging genes, as well as methods of identifying gene cliques, i.e. genes that co-express with a validated biomarker and exhibit correlation of expression with the validated biomarker, and thus may be substituted for that biomarker in an assay, are also provided. One skilled in the art would recognize that such substitutions may impact the association, for example the range of expression for the staging gene or gene subset associated with a particular stage may need to be adjusted.

In exemplary embodiments, expression levels of a staging gene from one of the staging gene subsets comprising a stromal group, apoptosis group, invasion group, metabolism group, signal transduction, and/or carbohydrate metabolism group may be used to determine the tumor stage. The stromal group includes at least one of the following: FAP, EFNB2, and SERPINB5, and genes that co-express with ANXA1, FAP, EFNB2 or SERPINB5. The apoptosis group includes ANXA1. The invasion group includes MMP11, and genes that co-express with MMP11. The metabolism group includes FABP4 and genes that co-express with FABP4. The carbohydrate metabolism group includes SI and genes that co-express with SI. The signal transduction group includes AKAP12. The calculation may be performed on a computer programmed to execute the gene expression analysis.

In exemplary embodiments, the molecular assay may involve expression levels for at least two staging genes. The staging genes, or staging gene subsets, may be weighted according to strength of association with tumor stage.

In exemplary embodiments, the expression of the staging genes may be thresholded, for example, based on a Cₜ value.

### DEFINITIONS

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described herein. For purposes of the invention, the following terms are defined below.

The terms “tumor” and “lesion” as used herein, refer to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer in the present disclosure include cancer of the gastrointestinal tract, such as invasive colorectal cancer or Dukes B (stage II) or Dukes C (stage III) colorectal cancer.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “colon cancer” and “colorectal cancer” are used interchangeably and in the broadest sense and refer to (1) all stages and all forms of cancer arising from epithelial cells of the large intestine and/or rectum and/or (2) all stages and all forms of cancer affecting the lining of the large intestine and/or rectum. In the staging systems used for classification of colorectal cancer, the colon and rectum are treated as one organ.

According to the tumor, node, metastasis (TNM) staging system of the American Joint Committee on Cancer (AJCC) (Greene et al. (eds.), AJCC Cancer Staging Manual. 6th Ed. New York, N.Y.: Springer; 2002), the various stages of colorectal cancer are defined as follows:

Tumor: T1: tumor invades submucosal T2: tumor invades muscularis propria; T3: tumor invades through the muscularis propria into the subserose, or into the pericolic or perirectal tissues; T4: tumor directly invades other organs or structures, and/or perforates.

Node: NO: no regional lymph node metastasis; N1: metastasis in 1 to 3 regional lymph nodes; N2: metastasis in 4 or more regional lymph nodes.

Metastasis: M0: mp distant metastasis; M1: distant metastasis present.

Stage groupings: Stage I: T1 NO MO; T2 NO MO; Stage II: T3 NO MO; T4 NO MO; Stage III: any T, N1-2; MO; Stage 1V: any T, any N, M1.

According to the Modified Duke Staging System, the various stages of colorectal cancer are defined as follows:

Stage A: the tumor penetrates into the mucosa of the bowel wall but not further. Stage B: tumor penetrates into and through the muscularis propria of the bowel wall; Stage C: tumor penetrates into but not through muscularis propria of the bowel wall, there is pathologic evidence of colorectal cancer in the lymph nodes; or tumor penetrates into and through the muscularis propria of the bowel wall, there is pathologic evidence of cancer in the lymph nodes; Stage D: tumor has spread beyond the confines of the lymph nodes, into other organs, such as the liver, lung or bone.

Prognostic factors are those variables related to the natural history of colorectal cancer, which influence the recurrence rates and outcome of patients once they have developed colorectal cancer. Clinical parameters that have been associated with a worse prognosis include, for example, lymph node involvement and increased tumor stage. Prognostic factors are frequently used to categorize patients into subgroups with different baseline relapse risks.

The term “prognosis” is used herein to refer to the prediction of the likelihood that a cancer patient will have a cancer-attributable death or progression, including recurrence, metastatic spread, and drug resistance, of a neoplastic disease, such as colon cancer.

The term “staging gene” is used herein to refer to a gene, the expression of which is correlated (associated), positively or negatively, with a particular tumor stage.

The methods of the present invention can be used clinically to determine the stage of a colorectal tumor, or provide confirmation of staging determined by pathology. The stage of a tumor is relevant to make treatment decisions, such as chemotherapy, surgical intervention, or both.

As used herein, the term “expression level” as applied to a gene refers to the normalized level of a gene product, e.g. the normalized value determined for the RNA expression level of a gene or for the polypeptide expression level of a gene.

The term “gene product” or “expression product” are used herein to refer to the RNA transcription products (transcripts) of the gene, including mRNA, and the polypeptide translation products of such RNA transcripts. A gene product can be, for example, an unspliced RNA, an mRNA, a splice variant mRNA, a microRNA, a fragmented RNA, a polypeptide, a post-translationally modified polypeptide, a splice variant polypeptide, etc.

The term “RNA transcript” as used herein refers to the RNA transcription products of a gene, including, for example, mRNA, an unspliced RNA, a splice variant mRNA, a microRNA, and a fragmented RNA.

Unless indicated otherwise, each gene name used herein corresponds to the Official Symbol assigned to the gene and provided by Entrez Gene (URL: www.ncbi.nlm.nih.gov/sites/entrez) as of the filing date of this application.

The terms “correlated” and “associated” are used interchangeably herein to refer to the strength of association between two measurements (or measured entities). The disclosure provides genes and gene subsets, the expression levels of which are associated with tumor stage. For example, the increased expression level of a gene may be positively correlated (positively associated) with an increased tumor stage, such as stage III. Such a positive correlation may be demonstrated statistically in various ways, e.g. by a low hazard ratio. In another example, the increased expression level of a gene may be negatively correlated (negatively associated) with an increased tumor stage. In that case, for example, the patient may have a stage II tumor. “Correlated” is also used herein to refer to the strength of association between the expression levels of two different genes, such that expression level of a first gene can be substituted with an expression level of a second gene in a given molecular assay in view of their correlation of expression. Such “correlated expression” of two genes that are substitutable in an assay usually gene expression levels that are positively correlated with one another, e.g., if increased expression of a first gene is positively correlated with a higher stage tumor, then the second gene that is co-expressed and exhibits correlated expression with the first gene is also positively correlated with higher stage.

The term “risk classification” means a level of risk (or likelihood) that a subject will experience a particular clinical outcome. A subject may be classified into a risk group or classified at a level of risk based on the methods of the present disclosure, e.g. high, medium, or low risk. A “risk group” is a group of subjects or individuals with a similar level of risk for a particular clinical outcome.

The term “long-term” survival is used herein to refer to survival for a particular time period, e.g., for at least 3 years, more preferably for at least 5 years.

The term “Recurrence-Free Interval (RFI)” is used herein to refer to the time (in years) from surgery or study randomization to first colon cancer recurrence or death due to recurrence of colorectal cancer.

The term “Overall Survival (OS)” is used herein to refer to the time (in years) from surgery or study randomization to death from any cause.

The term “Disease-Free Survival (DFS)” is used herein to refer to the time (in years) from surgery or study randomization to first colon cancer recurrence or death from any cause.

The term “Distant Recurrence-Free Interval (DRFI)” is used herein to refer to the time (in years) from surgery or study randomization to the first anatomically distant cancer recurrence.

The calculation of the measures listed above in practice may vary from study to study depending on the definition of events to be either censored or not considered.

The term “microarray” refers to an ordered arrangement of hybridizable array elements, e.g. oligonucleotide or polynucleotide probes, on a substrate.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons, are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNArDNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The term “Cₜ” as used herein refers to threshold cycle, the cycle number in quantitative polymerase chain reaction (qPCR) at which the fluorescence generated within a reaction well exceeds the defined threshold, i.e. the point during the reaction at which a sufficient number of amplicons have accumulated to meet the defined threshold.

The terms “threshold” or “thresholding” refer to a procedure used to account for non-linear relationships between gene expression measurements and clinical response as well as to further reduce variation in reported patient scores. When thresholding is applied, all measurements below or above a threshold are set to that threshold value. Non-linear relationship between gene expression and outcome could be examined using smoothers or cubic splines to model gene expression in Cox PH regression on recurrence free interval or logistic regression on recurrence status. Variation in reported patient scores could be examined as a function of variability in gene expression at the limit of quantitation and/or detection for a particular gene.

As used herein, the term “amplicon,” refers to pieces of DNA that have been synthesized using amplification techniques, such as polymerase chain reactions (PCR) and ligase chain reactions.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Fico11/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide, followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The terms “splicing” and “RNA splicing” are used interchangeably and refer to RNA processing that removes introns and joins exons to produce mature mRNA with continuous coding sequence that moves into the cytoplasm of an eukaryotic cell.

The term “co-expressed”, as used herein, refers to a statistical correlation between the expression level of one gene and the expression level of another gene. Pairwise co-expression may be calculated by various methods known in the art, e.g., by calculating Pearson correlation coefficients or Spearman correlation coefficients. Co-expressed gene cliques may also be identified using a graph theory. An analysis of co-expression may be calculated using normalized expression data. A gene is said to be co-expressed with a particular staging gene when the expression level of the gene exhibits a Pearson correlation coefficient greater than or equal to 0.6.

A “computer-based system” refers to a system of hardware, software, and data storage medium used to analyze information. The minimum hardware of a patient computer-based system comprises a central processing unit (CPU), and hardware for data input, data output (e.g., display), and data storage. An ordinarily skilled artisan can readily appreciate that any currently available computer-based systems and/or components thereof are suitable for use in connection with the methods of the present disclosure. The data storage medium may comprise any manufacture comprising a recording of the present information as described above, or a memory access device that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” or “computing means” references any hardware and/or software combination that will perform the functions required of it. For example, a suitable processor may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

As used herein, the term “surgery” applies to surgical methods undertaken for removal of cancerous tissue, including resection, laparotomy, colectomy (with or without lymphadenectomy), ablative therapy, endoscopic removal, excision, dissection, and tumor biopsy/removal. The tumor tissue or sections used for gene expression analysis may have been obtained from any of these methods.

### Gene Expression Methods Using Staging Genes and Staging Gene Subsets

The present disclosure provides methods to classify a tumor based on stage. The determination of stage is based on expression levels of one or more staging genes from particular staging gene subsets. For example, the expression level of one or more staging genes may be used to determine whether a colorectal tumor is a stage II or stage III tumor.

The gene subset identified herein as the “invasion group” includes genes involved in the breakdown of extracellular matrix and invasion. The invasion group includes, for example, STMY3 and genes that are co-expressed with STMY3.

The gene subset identified herein as the “stromal group” includes genes involved in tumors driven by multiple angiogenic factors that develop a wound-healing response. “Wound healing” refers to the process that a body uses to repair itself, and angiogenesis characterizes the proliferative phase of wound healing, during which new blood vessels are formed by vascular endothelial cells. The stromal group includes, for example, SERPINB5, ANXA1, FAP, and EFNB2, and genes that are co-expressed with SERPINB5, FAP, or EFNB2.

The gene subset identified as the “apoptosis group” includes genes involved in the process of programmed cell death. The apoptosis group includes ANXA1, and genes that are co-expressed with ANXA1. The gene subset identified herein as the “metabolism group” includes genes involved in fatty acid and glucose metabolism. The metabolism group includes, for example, FABP4 and genes that are co-expressed with FABP4.

The gene subset identified as the “carbohydrate metabolism group” includes genes involved in enterocyte differentiation and carbohydrate metabolosm. The carbohydrate metabolism group includes, for example, SI and genes that are co-expressed with SI.

The gene subset identified as the “signal transduction group” includes genes involved in cellular growth and signal transduction functions. The signal transduction group includes, for example, AKAP12 and genes that are co-expressed with AKAP12.

The gene subset identified as the “invasion group” includes genes involved in the breakdown of extracellular matrix. The invasion group includes, for example, MMP11 and genes that are co-expressed with MMP11.

Various technological approaches for determination of expression levels of the disclosed genes are set forth in this specification, including, without limitation, RT-PCR, microarrays, high-throughput sequencing, serial analysis of gene expression (SAGE) and Digital Gene Expression (DGE), which will be discussed in detail below. In particular aspects, the expression level of each gene may be determined in relation to various features of the expression products of the gene including exons, introns, protein epitopes and protein activity.

The expression levels of staging genes may be measured in tumor tissue. For example, the tumor tissue is obtained upon surgical removal or resection of the tumor, or by tumor biopsy. The expression level of staging genes may also be measured in tumor cells recovered from sites distant from the tumor, for example circulating tumor cells, body fluid (e.g., urine, blood, blood fraction, etc.).

The expression product that is assayed can be, for example, RNA or a polypeptide. The expression product may be fragmented. For example, the assay may use primers that are complementary to target sequences of an expression product and could thus measure full transcripts as well as those fragmented expression products containing the target sequence. Further information is provided in Tables A and B (inserted at the end of the specification).

The RNA expression product may be assayed directly or by detection of a cDNA product resulting from a PCR-based amplification method, e.g., quantitative reverse transcription polymerase chain reaction (qRT-PCR). (See e.g., U.S. Pub. No. US2006-0008809A1.) Polypeptide expression product may be assayed using immunohistochemistry (IHC). Further, both RNA and polypeptide expression products may also be is assayed using microarrays.

### Clinical Utility

The gene expression assay and associated information provided by the practice of the methods disclosed can be used to facilitate the identification of the stage of a patient's tumor. Given that tumor stage is a recognized prognostic factor, this information would assist physicians to make more well-informed treatment decisions, and to customize the treatment of colorectal cancer to the needs of individual patients, thereby maximizing the benefit of treatment and minimizing the exposure of patients to unnecessary treatments which may provide little or no significant benefits and often carry serious risks due to toxic side-effects.

Multi-analyte gene expression tests can be used to measure the expression level of one or more genes involved in each of several relevant physiologic processes or component cellular characteristics. The method disclosed herein may group the expression level values of staging genes. The grouping of staging genes may be performed at least in part based on knowledge of the contribution of those staging genes according to physiologic functions or component cellular characteristics, such as in the groups discussed above. The formation of groups (or staging gene subsets), in addition, can facilitate the mathematical weighting of the contribution of various expression levels to the identification of tumor stage. The weighting of a staging gene group representing a physiological process or component cellular characteristic can reflect the contribution of that process or characteristic to the pathology of the cancer and clinical outcome.

Optionally, given the relationship between stage and prognosis, the methods disclosed may be used to classify patients by risk, for example risk of recurrence. Patients can be partitioned into subgroups based on tumor stage, where all patients with tumors of a particular stage can be classified as belonging to a particular risk group. Thus, the values chosen will define subgroups of patients with respectively greater or lesser risk.

The utility of a staging gene marker in predicting tumor stage may not be unique to that marker. An alternative marker having an expression pattern that is parallel to that of a selected staging gene may be substituted for, or used in addition to, a staging gene. Due to the co-expression of such genes, substitution of expression level values should have little impact on the overall utility of the test. The closely similar expression patterns of two genes may result from involvement of both genes in the same process and/or being under common regulatory control in colon tumor cells. The present disclosure thus contemplates the use of such co-expressed genes or staging gene subsets as substitutes for, or in addition to, staging genes of the present disclosure.

In a specific embodiment, methods are disclosed herein for measuring the expression level of one or more staging genes to determine whether a colon cancer tumor is Stage II or Stage III. Such a test has utility in many areas, including in the development and appropriate use of drugs to treat Stage II and/or Stage III cancers of the colon and/or rectum, to stratify cancer patients for inclusion in (or exclusion from) clinical studies, to assist patients and physicians in making treatment decisions, provide economic benefits by targeting treatment based on personalized genomic profile, and the like. For example, the staging methods may be used on samples collected from patients in a clinical trial where tumor stage is relevant to the protocol, for example only patients with high grade tumors are included. Further, the methods disclosed herein may be used where a physician receives conflicting pathology reports regarding tumor stage, or seeks confirmation of stage for other reasons.

Staging of rectal tumors can be carried out based on similar criteria as for colon tumor staging, although there are some differences resulting, for example, from differences in the arrangement of the draining lymph nodes. As a result, Stage II/III rectal tumors bear a reasonable correlation to Stage II/III colon tumors as to their state of progression. As noted above, the rate of local recurrence and other aspects of prognosis differ between rectal cancer and colon cancer, and these differences may arise from difficulties in accomplishing total resection of rectal tumors. Nevertheless, there is no compelling evidence that there is a difference between colon cancer and rectal cancer as to the molecular characteristics of the respective tumors. Tests able to determine staging for rectal cancer patients have utility similar in nature as described for colon cancer tests and the same markers might well have utility in both cancer types.

### Methods of Assaying Expression Levels of a Gene Product

The methods and compositions of the present disclosure will employ, unless otherwise indicated, conventional techniques of molecular biology (including recombinant techniques), microbiology, cell biology, and biochemistry, which are within the skill of the art. Exemplary techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

Methods of gene expression profiling include methods based on hybridization analysis of polynucleotides, methods based on sequencing of polynucleotides, and proteomics-based methods. Exemplary methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription PCT (RT-PCR) (Weis et al., Trends in Genetics 8:263-264 (1992)). Antibodies may be employed that can recognize sequence-specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

Reverse Transcriptase PCR (RT-PCR)

Typically, mRNA is isolated from a test sample. The starting material is typically total RNA isolated from a human tumor, usually from a primary tumor. Optionally, normal tissues from the same patient can be used as an internal control. mRNA can be extracted from a tissue sample, e.g., from a sample that is fresh, frozen (e.g. fresh frozen), or paraffin-embedded and fixed (e.g. formalin-fixed).

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrés et al., BioTechniques 18:42044 (1995). In particular, RNA isolation can be performed using a purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

The sample containing the RNA is then subjected to reverse transcription to produce cDNA from the RNA template, followed by exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction.

PCR-based methods use a thermostable DNA-dependent DNA polymerase, such as a Taq DNA polymerase. For example, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction product. A third oligonucleotide, or probe, can be designed to facilitate detection of a nucleotide sequence of the amplicon located between the hybridization sites the two PCR primers. The probe can be detectably labeled, e.g., with a reporter dye, and can further be provided with both a fluorescent dye, and a quencher fluorescent dye, as in a Taqman® probe configuration. Where a Taqman® probe is used, during the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 384-well format on a thermocycler. The RT-PCR may be performed in triplicate wells with an equivalent of 2 ng RNA input per 10 μL-reaction volume. During amplification, laser-induced fluorescent signal is collected in real-time through fiber optics cables for all wells, and detected at the CCD. The system includes software for running the instrument and for analyzing the data.

5′-Nuclease assay data are initially expressed as a threshold cycle (“Cₜ”). Fluorescence values are recorded during every cycle and represent the amount of product amplified to that point in the amplification reaction. The threshold cycle (Cₜ) is generally described as the point when the fluorescent signal is first recorded as statistically significant.

To minimize errors and the effect of sample-to-sample variation, RT-PCR is usually performed using an internal standard. The ideal internal standard gene (also referred to as a reference gene) is expressed at a constant level among cancerous and non-cancerous tissue of the same origin (i.e., a level that is not significantly different among normal and cancerous tissues), and is not significantly unaffected by the experimental treatment (i.e., does not exhibit a significant difference in expression level in the relevant tissue as a result of exposure to chemotherapy). For example, reference genes useful in the methods disclosed herein should not exhibit significantly different expression levels in cancerous colon as compared to normal colon tissue. RNAs most frequently used to normalize patterns of gene expression are mRNAs for the housekeeping genes glyceraldehyde-3-phosphate-dehydrogenase (GAPDH) and β-actin. Exemplary reference genes used for normalization comprise one or more of the following genes: ATP5E, GPX1, PGK1, UBB, and VDAC2. Gene expression measurements can be normalized relative to the mean of one or more (e.g., 2, 3, 4, 5, or more) reference genes. Reference-normalized expression measurements can range from 0 to 15, where a one unit increase generally reflects a 2-fold increase in RNA quantity.

Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

The steps of a representative protocol for use in the methods of the present disclosure use fixed, paraffin-embedded tissues as the RNA source. mRNA isolation, purification, primer extension and amplification can be preformed according to methods available in the art. (see, e.g., Godfrey et al. J. Molec. Diagnostics 2: 84-91 (2000); Specht et al., Am. J. Pathol. 158: 419-29 (2001)). Briefly, a representative process starts with cutting about 10 μm thick sections of paraffin-embedded tumor tissue samples. The RNA is then extracted, and protein and DNA depleted from the RNA-containing sample. After analysis of the RNA concentration, RNA is reverse transcribed using gene specific primers followed by RT-PCR to provide for cDNA amplification products.

Design of Intron-Based PCR Primers and Probes

PCR primers and probes can be designed based upon exon or intron sequences present in the mRNA transcript of the gene of interest. Primer/probe design can be performed using publicly available software, such as the DNA BLAT software developed by Kent, W. J., Genome Res. 12(4):656-64 (2002), or by the BLAST software including its variations.

Where necessary or desired, repetitive sequences of the target sequence can be masked to mitigate non-specific signals. Exemplary tools to accomplish this include the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Rrawetz S, Misener S (eds) Bioinformatics Methods and Protocols: Methods in Molecular Biology. Humana Press, Totowa, N.J., pp 365-386).

Other factors that can influence PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases, and exhibit Tm's between 50 and 80° C., e.g. about 50 to 70° C.

For further guidelines for PCR primer and probe design see, e.g. Dieffenbach, C W. et al, “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods MoI. Biol. 70:520-527 (1997), the entire disclosures of which are hereby expressly incorporated by reference.

Tables A and B provide further information concerning the primer, probe, and amplicon sequences associated with the Examples disclosed herein.

MassARRAY® System

In MassARRAY-based methods, such as the exemplary method developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivarion of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

Other PCR-Based Methods

Further PCR-based techniques that can find use in the methods disclosed herein include, for example, BeadArray® technology (IIlumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression® (BADGE), using the commercially available LuminexlOO LabMAP® system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003).

Microarrays

Expression levels of a gene of interest can also be assessed using the microarray technique. In this method, polynucleotide sequences of interest (including cDNAs and oligonucleotides) are arrayed on a substrate. The arrayed sequences are then contacted under conditions suitable for specific hybridization with detectably labeled cDNA generated from mRNA of a test sample. As in the RT-PCR method, the source of mRNA typically is total RNA isolated from a tumor sample, and optionally from normal tissue of the same patient as an internal control or cell lines. mRNA can be extracted, for example, from frozen or archived paraffin-embedded and fixed (e.g. formalin-fixed) tissue samples.

For example, PCR amplified inserts of cDNA clones of a gene to be assayed are applied to a substrate in a dense array. Usually at least 10,000 nucleotide sequences are applied to the substrate. For example, the microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After washing under stringent conditions to remove non-specifically bound probes, the chip is scanned by confocal laser microscopy or by another detection method, such as a CCD camera. Quantitation of hybridization of each arrayed element allows for assessment of corresponding mRNA abundance.

With dual color fluorescence, separately labeled cDNA probes generated from two sources of RNA are hybridized pair wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et at, Proc. Natl. Acad. ScL USA 93(2):106-149 (1996)). Microarray analysis can be performed by commercially available equipment, following manufacturer's protocols, such as by using the Affymetrix GenChip® technology, or Incyte's microarray technology.

Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

Gene Expression Analysis by Nucleic Acid Sequencing

Nucleic acid sequencing technologies are suitable methods for analysis of gene expression. The principle underlying these methods is that the number of times a cDNA sequence is detected in a sample is directly related to the relative expression of the mRNA corresponding to that sequence. These methods are sometimes referred to by the term Digital Gene Expression (DGE) to reflect the discrete numeric property of the resulting data. Early methods applying this principle were Serial Analysis of Gene Expression (SAGE) and Massively Parallel Signature Sequencing (MPSS). See, e.g., S. Brenner, et al., Nature Biotechnology 18(6):630-634 (2000). More recently, the advent of “next-generation” sequencing technologies has made DGE simpler, higher throughput, and more affordable. As a result, more laboratories are able to utilize DGE to screen the expression of more genes in more individual patient samples than previously possible. See, e.g., J. Marioni, Genome Research 18(9):1509-1517 (2008); R. Morin, Genome Research 18(4):610-621 (2008); A. Mortazavi, Nature Methods 5(7):621-628 (2008); N. Cloonan, Nature Methods 5(7):613-619 (2008).

Isolating RNA from Body Fluids

Methods of isolating RNA for expression analysis from blood, plasma and serum (See for example, Tsui N B et al. (2002) 48, 1647-53 and references cited therein) and from urine (See for example, Boom R et al. (1990) J Clin Microbiol. 28, 495-503 and reference cited therein) have been described.

Immunohistochemistry

Immunohistochemistry methods are also suitable for detecting the expression levels of genes and applied to the method disclosed herein. Antibodies (e.g., monoclonal antibodies) that specifically bind a gene product of a gene of interest can be used in such methods. The antibodies can be detected by direct labeling of the antibodies themselves, for example, with radioactive labels, fluorescent labels, hapten′ labels such as, biotin, or an enzyme such as horse radish peroxidase or alkaline phosphatase. Alternatively, unlabeled primary antibody can be used in conjunction with a labeled secondary antibody specific for the primary antibody. Immunohistochemistry protocols and kits are well known in the art and are commercially available.

Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics.

General Description of the mRNA Isolation, Purification and Amplification

The steps of a representative protocol for profiling gene expression using fixed, paraffin-embedded tissues as the RNA source, including mRNA isolation, purification, primer extension and amplification are provided in various published journal articles. (See, e.g., T. E. Godfrey et al., J. Molec. Diagnostics 2: 84-91 (2000); K. Specht et al., Am. J. Pathol. 158: 419-29 (2001), M. Cronin, et al., Am J Pathol 164:35-42 (2004)). Briefly, a representative process starts with cutting a tissue sample section (e.g. about 10 μm thick sections of a paraffin-embedded tumor tissue sample). The RNA is then extracted, and protein and DNA are removed. After analysis of the RNA concentration, RNA repair is performed if desired. The sample can then be subjected to analysis, e.g., by reverse transcribed using gene specific promoters followed by RT-PCR.

### Statistical Analysis of Gene Expression Levels in Identification of Stage Genes

One skilled in the art will recognize that there are many statistical methods that may be used to determine whether there is a significant relationship between an parameter of interest (e.g., stage) and expression levels of a marker gene as described here. This relationship can be presented as a continuous value, or tumors may be stratified into stage groups (e.g., I-IV). For example, a Cox proportional hazards regression model may be used. One assumption of the Cox proportional hazards regression model is the proportional hazards assumption, i.e. the assumption that effect parameters multiply the underlying hazard. Assessments of model adequacy may be performed including, but not limited to, examination of the cumulative sum of martingale residuals. One skilled in the art would recognize that there are numerous statistical methods that may be used (e.g., Royston and Parmer (2002), smoothing spline, etc.) to fit a flexible parametric model using the hazard scale and the Weibull distribution with natural spline smoothing of the log cumulative hazards function. (See, P. Royston, M. Parmer, Statistics in Medicine 21(15:2175-2197 (2002).) The relationship between other clinical/pathologic covariates (e.g., number of nodes examined, tumor grade, MSI status, lymphatic or vascular invasion, etc.) may also be tested for significance.

### Coexpression Analysis

The present disclosure provides a method to determine tumor stage based on the expression of staging genes, or genes that co-express with particular staging genes. To perform particular biological processes, genes often work together in a concerted way, i.e. they are co-expressed. Co-expressed gene groups identified for a disease process like cancer can serve as biomarkers for tumor status and disease progression. Such co-expressed genes can be assayed in lieu of, or in addition to, assaying of the staging gene with which they are co-expressed.

One skilled in the art will recognize that many co-expression analysis methods now known or later developed will fall within the scope and spirit of the present invention. These methods may incorporate, for example, correlation coefficients, co-expression network analysis, clique analysis, etc., and may be based on expression data from RT-PCR, microarrays, sequencing, and other similar technologies. For example, gene expression clusters can be identified using pair-wise analysis of correlation based on Pearson or Spearman correlation coefficients. (See, e.g., Pearson K. and Lee A., Biometrika 2, 357 (1902); C. Spearman, Amer. J. Psychol 15:72-101 (1904); J. Myers, A. Well, Research Design and Statistical Analysis, p. 508 (2^{nd} Ed., 2003).) In general, a correlation coefficient of equal to or greater than 0.3 is considered to be statistically significant in a sample size of at least 20. (See, e.g., G. Norman, D. Streiner, Biostatistics: The Bare Essentials, 137-138 (3rd Ed. 2007).)

### Normalization of Expression Levels

The expression data used in the methods disclosed herein can be normalized. Normalization refers to a process to correct for (normalize away), for example, differences in the amount of RNA assayed and variability in the quality of the RNA used, to remove unwanted sources of systematic variation in Cₜ measurements, and the like. With respect to RT-PCR experiments involving archived fixed paraffin embedded tissue samples, sources of systematic variation are known to include the degree of RNA degradation relative to the age of the patient sample and the type of fixative used to store the sample. Other sources of systematic variation are attributable to laboratory processing conditions.

Assays can provide for normalization by incorporating the expression of certain normalizing genes, which genes do not significantly differ in expression levels under the relevant conditions. Exemplary normalization genes include housekeeping genes such as PGK1 and UBB. (See, e.g., E. Eisenberg, et al., Trends in Genetics 19(7):362-365 (2003).) Normalization can be based on the mean or median signal (C_{T}) of all of the assayed genes or a large subset thereof (global normalization approach). In general, the normalizing genes, also referred to as reference genes should be genes that are known not to exhibit significantly different expression in colorectal cancer as compared to non-cancerous colorectal tissue, and are not significantly affected by various sample and process conditions, thus provide for normalizing away extraneous effects.

Unless noted otherwise, normalized expression levels for each mRNA/tested tumor/patient will be expressed as a percentage of the expression level measured in the reference set. A reference set of a sufficiently high number of tumors yields a distribution of normalized levels of each mRNA species. The level measured in a particular tumor sample to be analyzed falls at some percentile within this range, which can be determined by methods well known in the art.

In exemplary embodiments, one or more of the following genes are used as references by which the expression data is normalized: ATP5E, GPX1, PGK1, UBB, and VDAC2. The calibrated weighted average Cₜ measurements for each of the prognostic and predictive genes may be normalized relative to the mean of five or more reference genes.

Those skilled in the art will recognize that normalization may be achieved in numerous ways, and the techniques described above are intended only to be exemplary, not exhaustive.

### Kits of the Invention

The materials for use in the methods of the present invention are suited for preparation of kits produced in accordance with well known procedures. The present disclosure thus provides kits comprising agents, which may include gene-specific or gene-selective probes and/or primers, for quantifying the expression of the disclosed genes for predicting prognostic outcome or response to treatment. Such kits may optionally contain reagents for the extraction of RNA from tumor samples, in particular fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microliter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more probes and primers of the present invention (e.g., appropriate length poly(T) or random primers linked to a promoter reactive with the RNA polymerase). Mathematical algorithms used to estimate or quantify prognostic or predictive information are also properly potential components of kits.

### Reports

The methods of this invention, when practiced for commercial diagnostic purposes, generally produce a report or summary of information obtained from the herein-described methods. For example, a report may include information concerning expression levels of staging genes, identification of the tumor stage, or classification of the tumor or the patient according to prognosis based on tumor stage and/or other information. The methods and reports of this invention can further include storing the report in a database. The method can create a record in a database for the subject and populate the record with data. The report may be a paper report, an auditory report, or an electronic record. The report may be displayed and/or stored on a computing device (e.g., handheld device, desktop computer, smart device, website, etc.). It is contemplated that the report is provided to a physician and/or the patient. The receiving of the report can further include establishing a network connection to a server computer that includes the data and report and requesting the data and report from the server computer.

### Computer Program

The values from the assays described above, such as expression data, can be calculated and stored manually. Alternatively, the above-described steps can be completely or partially performed by a computer program product. The present invention thus provides a computer program product including a computer readable storage medium having a computer program stored on it. The program can, when read by a computer, execute relevant calculations based on values obtained from analysis of one or more biological sample from an individual (e.g., gene expression levels, normalization, thresholding, and conversion of values from assays to a score and/or text or graphical depiction of tumor stage and related information). The computer program product has stored therein a computer program for performing the calculation.

The present disclosure provides systems for executing the program described above, which system generally includes: a) a central computing environment; b) an input device, operatively connected to the computing environment, to receive patient data, wherein the patient data can include, for example, expression level or other value obtained from an assay using a biological sample from the patient, or microarray data, as described in detail above; c) an output device, connected to the computing environment, to provide information to a user (e.g., medical personnel); and d) an algorithm executed by the central computing environment (e.g., a processor), where the algorithm is executed based on the data received by the input device, and wherein the algorithm calculates an expression score, thresholding, or other functions described herein. The methods provided by the present invention may also be automated in whole or in part.

All aspects of the present invention may also be practiced such that a limited number of additional genes that are co-expressed with the disclosed genes, for example as evidenced by statistically meaningful Pearson and/or Spearman correlation coefficients, are included in a test in addition to and/or in place of disclosed genes.

Having described the invention, the same will be more readily understood through reference to the following Examples, which are provided by way of illustration, and are not intended to limit the invention in any way.

### EXAMPLE 1

### Gene Expression Analysis for Tumor Stage

Patients and Samples

Tumor tissue samples from four cohorts of patients with stage II or stage III colon cancer treated with surgery alone or surgery plus 5-FU/LV-based chemotherapy form the basis for this report. Further details concerning the Cleveland Clinic Foundation (CCF) and National Surgical Adjuvant Breast and Bowel Project (NSABP) protocols C-01, C-02, C-03, and C-04 are available in C. Allegra, J Clin Oncology 21(2):241-250 (2003) and U.S. Ser. No. 12/772,136, filed Apr. 30, 2010, the contents of which are incorporated herein by reference. Gene expression measurements were obtained from archived, formalin-fixed, paraffin-embedded (FPE) colon tumor tissue.

Statistical Analysis

The relationship between gene expression and tumor stage was investigated across four studies. Two separate analyses were conducted. In the first analysis, the stage II patients were restricted to those who had at least 12 nodes examined, to minimize the chances of under-staging in stage II. The second analysis included all stage II patients, including those who had fewer than 12 nodes examined Table 1 shows the number of patients in each stage for each study.

TABLE 1
Numbers of Patients Analyzed, by Study and Stage
(Excluding Stage II Patients with <12 Nodes Examined)

| Stage | C-01/C-02 | C-04 | CCF | C-06 | Total |
|---|---|---|---|---|---|
| II | 62 | 66 | 387 | 119 | 634 |
| (≧12 nodes examined) | | | | | |
| III | 139 | 171 | 261 | 273 | 844 |
| Total | 201 | 237 | 648 | 392 | 1,478 |

In each study, two-sample t-tests were used to compare mean expression levels between patients with stage II and stage III colon cancer for each of the 375 genes that were studied in all 4 studies. Five of the 375 genes had significant (two-sided p<0.05) differences in mean expression between stage II and stage III patients in all 4 studies. Table 2 displays the study-specific and stage-specific mean expression levels of each of the 5 genes, along with the p-values for the comparison between stages for each of the 4 studies. Table 3

TABLE 2
Stage-specific Mean Gene Expression Levels in 4 Studies
(Excluding Stage II Patients with <12 Nodes Examined)

| | | Study | | | | | | | |
|---|---|---|---|---|---|---|---|---|---|
| | | C-01/C-02 | | C-04 | | CCF | | C-06 | |
| | | Stage | | | | | | | |
| Gene | | II | III | II | III | II | III | II | III |
| EFNB2 | N | 62 | 138 | 66 | 171 | 387 | 261 | 119 | 273 |
| NM_004093 | mean | 4.11 | 4.55 | 4.12 | 4.61 | 4.75 | 4.93 | 4.57 | 4.86 |
| | p- | 0.002 | | <0.001 | | 0.013 | | 0.001 | |
| | value | | | | | | | | |
| FABP4 | N | 62 | 139 | 66 | 171 | 387 | 260 | 119 | 273 |
| NM_001442 | mean | 3.65 | 4.05 | 3.95 | 4.65 | 3.06 | 3.27 | 3.43 | 3.85 |
| | p- | 0.042 | | 0.005 | | 0.009 | | 0.003 | |
| | value | | | | | | | | |
| SERPINB5 | N | 62 | 139 | 66 | 171 | 387 | 261 | 119 | 273 |
| NM_002639 | mean | 3.52 | 4.20 | 3.96 | 4.48 | 4.43 | 4.70 | 4.39 | 4.85 |
| | p- | <0.001 | | 0.008 | | 0.032 | | 0.003 | |
| | value | | | | | | | | |
| SI | N | 62 | 139 | 66 | 171 | 387 | 261 | 119 | 273 |
| NM_001041 | mean | 2.56 | 2.85 | 2.54 | 2.70 | 2.66 | 2.79 | 2.74 | 2.94 |
| | p- | <0.001 | | 0.007 | | 0.017 | | 0.008 | |
| | value | | | | | | | | |
| MMP11 | N | 2 | 139 | 66 | 171 | 387 | 260 | 119 | 273 |
| NM_005940 | mean | .68 | 5.40 | 5.57 | 6.31 | 5.98 | 6.27 | 6.75 | 7.26 |

| | | | | |
|---|---|---|---|---|
| p- | 0.001 | <0.001 | 0.011 | 0.001 |
| value | | | | |

The number of patients in each stage for each study are depicted in Table 3, for the case where stage II patients with <12 nodes examined were included with the other stage II patients.

TABLE 3
Numbers of Patients Analyzed, by Study and Stage
(Including Stage II Patients with <12 Nodes Examined)

| Stage | C-01/C-02 | C-04 | CCF | C-06 | Total |
|---|---|---|---|---|---|
| II | 131 | 137 | 504 | 235 | 1,007 |
| III | 139 | 171 | 261 | 273 | 844 |
| Total | 270 | 308 | 765 | 508 | 1,851 |

In each study, two-sample t-tests were used to compare mean expression levels between patients with stage II and stage III colon cancer for each of the 375 genes that were studied in all 4 studies. Six of the 375 genes had significant (two-sided p<0.05) differences in mean expression between stage II and stage III patients in all 4 studies. Table 4 below displays the study-specific and stage-specific mean expression levels of each of the 6 genes, along with the p-values for the comparison between stages for each of the 4 studies.

TABLE 4
Stage-specific Mean Gene Expression Levels in 4 Development Studies
(Including Stage II Patients with <12 Nodes Examined)

| | | Study | | | | | | | |
|---|---|---|---|---|---|---|---|---|---|
| | | C-01/C-02 | | C-04 | | CCF | | C-06 | |
| | | Stage | | | | | | | |
| Gene | | II | III | II | III | II | III | II | III |
| AKAP12 | N | 131 | 139 | 137 | 171 | 504 | 261 | 235 | 273 |
| NM_005100 | mean | 5.27 | 5.55 | 5.46 | 5.87 | 5.08 | 5.41 | 6.07 | 6.28 |
| | p- | 0.045 | | 0.001 | | <0.001 | | 0.014 | |
| | value | | | | | | | | |
| ANXA1 | N | 131 | 139 | 137 | 171 | 504 | 261 | 235 | 273 |
| NM_000700 | mean | 7.24 | 7.49 | 7.36 | 7.59 | 7.48 | 7.74 | 7.41 | 7.63 |
| | p- | 0.022 | | 0.025 | | <0.001 | | 0.002 | |
| | value | | | | | | | | |
| EFNB2 | N | 130 | 138 | 137 | 171 | 504 | 261 | 235 | 273 |
| NM_004093 | mean | 4.31 | 4.55 | 4.26 | 4.61 | 4.74 | 4.93 | 4.66 | 4.86 |
| | p- | 0.035 | | <0.001 | | 0.007 | | 0.004 | |
| | value | | | | | | | | |
| FAP | N | 131 | 139 | 137 | 171 | 504 | 261 | 235 | 273 |
| NM_004460 | mean | 5.91 | 6.20 | 5.95 | 6.25 | 5.99 | 6.19 | 5.98 | 6.15 |
| | p- | 0.032 | | 0.014 | | 0.013 | | 0.025 | |
| | value | | | | | | | | |
| SERPINB5 | n | 131 | 39 | 137 | 171 | 504 | 261 | 235 | 273 |
| NM_002639 | mean | .69 | .20 | 3.99 | 4.48 | 4.40 | 4.70 | 4.36 | 4.85 |
| | p- | 0.001 | | 0.002 | | 0.010 | | <0.001 | |
| | value | | | | | | | | |
| SI | n | 131 | 39 | 137 | 171 | 504 | 261 | 235 | 273 |
| NM_001041 | Mean | 2.58 | 2.85 | 2.58 | 2.70 | 2.68 | 2.79 | 2.81 | 2.94 |

| | | | | |
|---|---|---|---|---|
| p- | <0.001 | 0.020 | 0.038 | 0.042 |
| value | | | | |

TABLE A

| Gene | Accession | Reagt | Sequence | SEQ ID NO |
|---|---|---|---|---|
| AKAP12 | NM_005100.2 | FPr | TAGAGAGCCCCTGACAATCC | SEQ ID NO: 1 |
| | | Probe | TGGCTCTAGCTCCTGATGAAGCCTC | SEQ ID NO: 2 |
| | | RPr | GGTTGGTCTTGGAAAGAGGA | SEQ ID NO: 3 |
| ANXA1 | NM_000700.1 | FPr | GCCCCTATCCTACCTTCAATCC | SEQ ID NO: 4 |
| | | Probe | TCCTCGGATGTCGCTGCCT | SEQ ID NO: 5 |
| | | RPr | CCTTTAACCATTATGGCCTTATGC | SEQ ID NO: 6 |
| EFNB2 | NM_004093.2 | FPr | TGACATTATCATCCCGCTAAGGA | SEQ ID NO: 7 |
| | | Probe | CGGACAGCGTCTTCTGCCCTCACT | SEQ ID NO: 8 |
| | | RPr | GTAGTCCCCGCTGACCTTCTC | SEQ ID NO: 9 |
| FABP4 | NM_001442.1 | FPr | GCTTTGCCACCAGGAAAGT | SEQ ID NO: 10 |
| | | Probe | CTGGCATGGCCAAACCTAACATGA | SEQ ID NO: 11 |
| | | RPr | CATCCCCATTCACACTGATG | SEQ ID NO: 12 |
| FAP | NM_004460.2 | FPr | CTGACCAGAACCACGGCT | SEQ ID NO: 13 |
| | | Probe | CGGCCTGTCCACGAACCACTTATA | SEQ ID NO: 14 |
| | | RPr | GGAAGTGGGTCATGTGGG | SEQ ID NO: 15 |
| Maspin | NM_002639.1 | FPr | CAGATGGCCACTTTGAGAACATT | SEQ ID NO: 16 |
| | | Probe | AGCTGACAACAGTGTGAACGACCAGACC | SEQ ID NO: 17 |
| | | RPr | GGCAGCATTAACCACAAGGATT | SEQ ID NO: 18 |
| MMP1 | NM_002421.2 | FPr | GGGAGATCATCGGGACAACTC | SEQ ID NO: 19 |
| | | Probe | AGCAAGATTTCCTCCAGGTCCATCAAAAGG | SEQ ID NO: 20 |
| | | RPr | GGGCCTGGTTGAAAAGCAT | SEQ ID NO: 21 |
| SERPINB5 | NM_002639.1 | FPr | CAGATGGCCACTTTGAGAACATT | SEQ ID NO: 22 |
| | | Probe | AGCTGACAACAGTGTGAACGACCAGACC | SEQ ID NO: 23 |
| | | RPr | GGCAGCATTAACCACAAGGATT | SEQ ID NO: 24 |
| SI | NM_001041.1 | FPr | AACGGACTCCCTCAATTTGT | SEQ ID NO: 25 |
| | | Probe | TGTCCATGGTCATGCAAATCTTGC | SEQ ID NO: 26 |
| | | RPr | GAAATTGCAGGGTCCAAGAT | SEQ ID NO: 27 |
| STMY3 | NM_005940.2 | FPr | CCTGGAGGCTGCAACATACC | SEQ ID NO: 28 |
| | | Probe | ATCCTCCTGAAGCCCTTTTCGCAGC | SEQ ID NO: 29 |
| | | RPr | TACAATGGCTTTGGAGGATAGCA | SEQ ID NO: 30 |

TABLE B

| | | | SEQ ID |
|---|---|---|---|
| Gene | Locus Link | Sequence | NO |
| AKAP12 | NM_005100.2 | TAGAGAGCCCCTGACAATCCTGAGGCTTCATCAGGAGC | SEQ ID |
| | | TAGAGCCATTTAACATTTCCTCTTTCCAAGACCAACC | NO: 31 |
| ANXA1 | NM_000700.1 | GCCCCTATCCTACCTTCAATCCATCCTCGGATGT | SEQ ID |
| | | CGCTGCCTTGCATAAGGCCATAATGGTTAAAGG | NO: 32 |
| EFNB2 | NM_004093.2 | TGACATTATCATCCCGCTAAGGACTGCGGACAGCGTC | SEQ ID |
| | | TTCTGCCCTCACTACGAGAAGGTCAGCGGGGACTAC | NO: 33 |
| FABP4 | NM_001442.1 | GCTTTGCCACCAGGAAAGTGGCTGGCATGGCCA | SEQ ID |
| | | AACCTAACATGATCATCAGTGTGAATGGGGATG | NO: 34 |
| FAP | NM_004460.2 | CTGACCAGAACCACGGCTTATCCGGCCTGTCCA | SEQ ID |
| | | CGAACCACTTATACACCCACATGACCCACTTCC | NO: 35 |
| Maspin | NM_002639.1 | CAGATGGCCACTTTGAGAACATTTTAGCTGACAACAGTG | SEQ ID |
| | | TGAACGACCAGACCAAAATCCTTGTGGTTAATGCTGCC | NO: 36 |
| MMP1 | NM_002421.2 | GGGAGATCATCGGGACAACTCTCCTTTTGATGGACC | SEQ ID |
| | | TGGAGGAAATCTTGCTCATGCTTTTCAACCAGGCCC | NO: 37 |
| SERPINB5 | NM_002639.1 | CAGATGGCCACTTTGAGAACATTTTAGCTGACAACAGTG | SEQ ID |
| | | TGAACGACCAGACCAAAATCCTTGTGGTTAATGCTGCC | NO: 38 |
| SI | NM_001041.1 | AACGGACTCCCTCAATTTGTGCAAGATTTGCATGACCAT | SEQ ID |
| | | GGACAGAAATATGTCATCATCTTGGACCCTGCAATTTC | NO: 39 |
| STMY3 | NM_005940.2 | CCTGGAGGCTGCAACATACCTCAATCCTGT | SEQ ID |
| | | CCCAGGCCGGATCCTCCTGAAGCCCTTTTC | NO: 40 |
| | | GCAGCACTGCTATCCTCCAAAGCCATTGTA | |

What is claimed is:

1. A method of determining whether a patient has stage II or stage III colorectal cancer comprising:
a. assaying a level of at least one RNA transcript, or an expression product thereof, in a tumor sample obtained from the patient, wherein the at least one RNA transcript, or an expression product thereof, is selected from EFNB2, FABP4, SERPINB5, SI, MMP11, AKAP12, ANXA1, and FAP;
b. normalizing the level of the at least one RNA transcript, or an expression product thereof, to obtain a normalized expression level; and
c. determining the colorectal tumor stage of the patient, wherein an increased normalized expression level correlates with the patient having stage III colorectal cancer.

2. The method of claim 1, wherein the tumor sample is formalin-fixed, paraffin-embedded colon tumor tissue.

3. The method of claim 1, wherein the level of the at least one RNA transcript is assayed.

4. The method of claim 3, wherein the assaying is done using reverse transcription polymerase chain reaction (RT-PCR).

5. The method of claim 1, further comprising creating a report based on the normalized expression level.

6. The method of claim 1, wherein the at least one RNA transcript, or an expression product thereof, is EFNB2, FABP4, SERPINB5, SI, and MMP11.

7. A method of determining whether a patient has stage II or stage III colorectal cancer comprising:
a. assaying a level of at least one RNA transcript, or an expression product thereof, in a tumor sample obtained from the patient, wherein the at least one RNA transcript, or an expression product thereof, is an expression product of a gene from a gene subset selected from an invasion group, a stromal group, an apoptosis group, a metabolism group, a carbohydrate metabolism group, and a signal transduction group;
b. normalizing the level of the at least one RNA transcript, or an expression product thereof, to obtain a normalized expression level; and
c. determining the colorectal tumor stage of the patient, wherein an increased normalized expression level correlates with the patient having stage III colorectal cancer.

8. The method of claim 7, wherein the invasion group comprises MMP11, and co-expressed genes thereof.

9. The method of claim 7, wherein the stromal group comprises SERPINB5, ANXA1, FAP, and EFNB2, and co-expressed genes thereof.

10. The method of claim 7, wherein the apoptosis group comprises ANXA1, and co-expressed genes thereof.

11. The method of claim 7, wherein the metabolism group comprises FABP4, and co-expressed genes thereof.

12. The method of claim 7, wherein the carbohydrate metabolism group comprises SI, and co-expressed genes thereof.

13. The method of claim 7, wherein the signal transduction group comprises AKAP12, and co-expressed genes thereof.

14. The method of claim 7, wherein the tumor sample is formalin-fixed, paraffin-embedded colon tumor tissue.

15. The method of claim 7, wherein the level of the at least one RNA transcript is assayed.

16. The method of claim 15, wherein the assaying is done using reverse transcription polymerase chain reaction (RT-PCR).

17. The method of claim 7, further comprising creating a report based on the normalized expression level.


SEQUENCE LISTING (40 sequences)

SEQ ID NO: 1 - DNA, 20 bp, Artificial Sequence
  Synthetic oligonucleotide
tagagagcccctgacaatcc

SEQ ID NO: 2 - DNA, 25 bp, Artificial Sequence
  Synthetic oligonucleotide
tggctctagctcctgatgaagcctc

SEQ ID NO: 3 - DNA, 20 bp, Artificial Sequence
  Synthetic oligonucleotide
ggttggtcttggaaagagga

SEQ ID NO: 4 - DNA, 22 bp, Artificial Sequence
  Synthetic oligonucleotide
gcccctatcctaccttcaatcc

SEQ ID NO: 5 - DNA, 19 bp, Artificial Sequence
  Synthetic oligonucleotide
tcctcggatgtcgctgcct

SEQ ID NO: 6 - DNA, 24 bp, Artificial Sequence
  Synthetic oligonucleotide
cctttaaccattatggccttatgc

SEQ ID NO: 7 - DNA, 23 bp, Artificial Sequence
  Synthetic oligonucleotide
tgacattatcatcccgctaagga

SEQ ID NO: 8 - DNA, 24 bp, Artificial Sequence
  Synthetic oligonucleotide
cggacagcgtcttctgccctcact

SEQ ID NO: 9 - DNA, 21 bp, Artificial Sequence
  Synthetic oligonucleotide
gtagtccccgctgaccttctc

SEQ ID NO: 10 - DNA, 19 bp, Artificial Sequence
  Synthetic oligonucleotide
gctttgccaccaggaaagt

SEQ ID NO: 11 - DNA, 24 bp, Artificial Sequence
  Synthetic oligonucleotide
ctggcatggccaaacctaacatga

SEQ ID NO: 12 - DNA, 20 bp, Artificial Sequence
  Synthetic oligonucleotide
catccccattcacactgatg

SEQ ID NO: 13 - DNA, 18 bp, Artificial Sequence
  Synthetic oligonucleotide
ctgaccagaaccacggct

SEQ ID NO: 14 - DNA, 24 bp, Artificial Sequence
  Synthetic oligonucleotide
cggcctgtccacgaaccacttata

SEQ ID NO: 15 - DNA, 18 bp, Artificial Sequence
  Synthetic oligonucleotide
ggaagtgggtcatgtggg

SEQ ID NO: 16 - DNA, 23 bp, Artificial Sequence
  Synthetic oligonucleotide
cagatggccactttgagaacatt

SEQ ID NO: 17 - DNA, 28 bp, Artificial Sequence
  Synthetic oligonucleotide
agctgacaacagtgtgaacgaccagacc

SEQ ID NO: 18 - DNA, 22 bp, Artificial Sequence
  Synthetic oligonucleotide
ggcagcattaaccacaaggatt

SEQ ID NO: 19 - DNA, 21 bp, Artificial Sequence
  Synthetic oligonucleotide
gggagatcatcgggacaactc

SEQ ID NO: 20 - DNA, 30 bp, Artificial Sequence
  Synthetic oligonucleotide
agcaagatttcctccaggtccatcaaaagg

SEQ ID NO: 21 - DNA, 19 bp, Artificial Sequence
  Synthetic oligonucleotide
gggcctggttgaaaagcat

SEQ ID NO: 22 - DNA, 23 bp, Artificial Sequence
  Synthetic oligonucleotide
cagatggccactttgagaacatt

SEQ ID NO: 23 - DNA, 28 bp, Artificial Sequence
  Synthetic oligonucleotide
agctgacaacagtgtgaacgaccagacc

SEQ ID NO: 24 - DNA, 22 bp, Artificial Sequence
  Synthetic oligonucleotide
ggcagcattaaccacaaggatt

SEQ ID NO: 25 - DNA, 20 bp, Artificial Sequence
  Synthetic oligonucleotide
aacggactccctcaatttgt

SEQ ID NO: 26 - DNA, 24 bp, Artificial Sequence
  Synthetic oligonucleotide
tgtccatggtcatgcaaatcttgc

SEQ ID NO: 27 - DNA, 20 bp, Artificial Sequence
  Synthetic oligonucleotide
gaaattgcagggtccaagat

SEQ ID NO: 28 - DNA, 20 bp, Artificial Sequence
  Synthetic oligonucleotide
cctggaggctgcaacatacc

SEQ ID NO: 29 - DNA, 25 bp, Artificial Sequence
  Synthetic oligonucleotide
atcctcctgaagcccttttcgcagc

SEQ ID NO: 30 - DNA, 23 bp, Artificial Sequence
  Synthetic oligonucleotide
tacaatggctttggaggatagca

SEQ ID NO: 31 - DNA, 75 bp, Artificial Sequence
  Synthetic oligonucleotide
tagagagcccctgacaatcctgaggcttcatcaggagctagagccatttaacatttcctctttccaagaccaacc

SEQ ID NO: 32 - DNA, 67 bp, Artificial Sequence
  Synthetic oligonucleotide
gcccctatcctaccttcaatccatcctcggatgtcgctgccttgcataaggccataatggttaaagg

SEQ ID NO: 33 - DNA, 73 bp, Artificial Sequence
  Synthetic oligonucleotide
tgacattatcatcccgctaaggactgcggacagcgtcttctgccctcactacgagaaggtcagcggggactac

SEQ ID NO: 34 - DNA, 66 bp, Artificial Sequence
  Synthetic oligonucleotide
gctttgccaccaggaaagtggctggcatggccaaacctaacatgatcatcagtgtgaatggggatg

SEQ ID NO: 35 - DNA, 66 bp, Artificial Sequence
  Synthetic oligonucleotide
ctgaccagaaccacggcttatccggcctgtccacgaaccacttatacacccacatgacccacttcc

SEQ ID NO: 36 - DNA, 77 bp, Artificial Sequence
  Synthetic oligonucleotide
cagatggccactttgagaacattttagctgacaacagtgtgaacgaccagaccaaaatccttgtggttaatgctgcc

SEQ ID NO: 37 - DNA, 72 bp, Artificial Sequence
  Synthetic oligonucleotide
gggagatcatcgggacaactctccttttgatggacctggaggaaatcttgctcatgcttttcaaccaggccc

SEQ ID NO: 38 - DNA, 77 bp, Artificial Sequence; identical to SEQ ID NO: 36
  Synthetic oligonucleotide

SEQ ID NO: 39 - DNA, 77 bp, Artificial Sequence
  Synthetic oligonucleotide
aacggactccctcaatttgtgcaagatttgcatgaccatggacagaaatatgtcatcatcttggaccctgcaatttc

SEQ ID NO: 40 - DNA, 90 bp, Artificial Sequence
  Synthetic oligonucleotide
cctggaggctgcaacatacctcaatcctgtcccaggccggatcctcctgaagcccttttcgcagcactgctatcctccaaagccattgta